(19) United States
(12) Patent Application Publication
(10) Pub. No.: US 2025/0205165 A1
(43) Pub. Date: Jun. 26, 2025

(54) THERAPEUTIC VESICLES AND METHODS OF PROCESSING THE SAME

(71) Applicant: TheraXyte Bioscience Hong Kong Limited, Hong Kong (CN)

(72) Inventors: Tong ZHAO, Hong Kong (CN); Ruining WANG, Hong Kong (CN)

(21) Appl. No.: 18/849,254

(22) PCT Filed: Mar. 22, 2023
(86) PCT No.: PCT/CN2023/083033

§ 371 Date: Sep. 20, 2024

(30) Foreign Application Priority Data
Mar. 22, 2022 (WO) PCT/CN2022/082187

(51) Int. Cl. A61K 9/50; C12N 5/071; C12N 9/22; C12N 15/113

(52) U.S. Cl. CPC A61K 9/5089; A61K 9/5068; C12N 5/063; C12N 9/22; C12N 15/113; C12N 2310/11; C12N 2310/20

(57) ABSTRACT

Provided are extracellular vesicles, and methods of preparing the extracellular vesicles in the presence of a detergent.

### CROSS-REFERENCE TO RELATED APPLICATIONS

This application claims the priority of the International Application No. PCT/CN 2022/082187, filed on Mar. 22, 2022, the entire contents of which are incorporated herein by reference.

### TECHNICAL FIELD

The disclosure relates to therapeutic vesicles and methods of processing thereof.

### BACKGROUND OF THE INVENTION

Extracellular vesicles (EVs) are naturally derived particles secreted from cells and present in most organisms. Recently discovered vital biological function of EVs includes facilitation of the intercellular communication process by acting as cargo-ensembles for transporting essential cellular components (i.e., soluble proteins or active enzymes, lipids, and nucleic acids such as mRNAs, micro-RNAs, long non-coding RNAs, and metabolites). Such transportation capability of EVs prompted the exploration of utilizing EVs for delivering an agent (e.g., a therapeutic agent) to or within a target cell.

Compared to other well-known synthetic drug delivery vehicles (e.g., liposome, lipid nanoparticles or viral vectors, etc.), EVs provide numerous advantages as drug carriers due to their characteristics of being: i) natural secretomes from cells for short- or long-distance intercellular communication; ii) expected to have tropism for specific organs or cells via binding to certain surface receptors; iii) superior in cargo trafficking efficiency due to their multiple cell uptake routes which may include endocytosis, phagocytosis, micropinocytosis, or direct fusion with the recipient cell membranes; and iv) able to avoid immunological clearance owing to the intrinsic nature of the carrier.

However, despite the above-listed benefits, using currently available EVs as agent-delivering carriers suffers from a number of drawbacks relating to potential toxicity and relatively low loading efficiency, in which ultimately results in lower therapeutic efficacy. In particular, the known active loading strategies for EVs include electroporation, sonication, freeze-thawing, and chemicals assisted loading, in where each one of these methods poses concerns in terms of chemical or biological toxicity and subpar loading efficiency.

Thus, there is a need in the art to provide improved EVs incorporation methods for producing EVs with superior loading capacity as well as reduced side-effects for their use in delivering therapeutic agents or any molecules of interest.

### SUMMARY OF THE INVENTION

Disclosed herein are methods of manufacturing extracellular vesicles. Also disclosed herein is a kit for making extracellular vesicles in the presence of at least one detergent and one or more detergent-removal agents.

In one aspect, the disclosure provides a method for processing extracellular vesicles (EVs). The method comprises the steps of contacting a biological sample comprising a plurality of extracellular vesicles with a cargo molecule and a detergent to form a detergent-mixture solution; and removing the detergent from the detergent-mixture solution to obtain a plurality of cargo-loaded extracellular vesicles.

In some cases, the biological sample that comprises extracellular vesicles may contact the cargo molecule and the detergent by adding the cargo molecule and the detergent simultaneously. In some cases, the biological sample that comprises extracellular vesicles may contact the cargo molecule and the detergent by adding the cargo molecule and the detergent sequentially. In some cases, the method comprises a step of contacting the biological sample comprising a plurality of EVs with the cargo molecule and the detergent by adding the cargo molecule prior to adding the detergent. In other cases, the method comprises a step of contacting the biological sample comprising a plurality of EVs with the cargo molecule and the detergent by adding the detergent prior to adding the cargo molecule.

In some cases, the detergent comprises a surfactant. In some cases, the surfactant is a non-ionic surfactant. In these cases, the surfactant comprises a hydrophilic group and a hydrophobic group. In some cases, the hydrophilic group of the surfactant comprises a polyethylene oxide chain. In some cases, the surfactant is 2-[4-(2,4,4-trimethylpentan-2-yl)phenoxy] ethanol. In some cases, the surfactant is dodecyloctaglycol or octaethylene glycol monododecyl ether.

In some cases, the cargo molecule comprises an active pharmaceutical ingredient (API). In some cases, the cargo molecule comprises a small molecule therapeutics. In some cases, the cargo molecule comprises a polypeptide, protein, lipid, nucleic acid, carbohydrate, lipid, metabolite, or any combinations thereof. In some cases, the nucleic acid comprises DNA. In some cases, the nucleic acid comprises peptide nucleic acids (PNAs). In some cases, the nucleic acid comprises RNA. In some cases, the RNA is selected from the group consisting of, mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (IncRNA). In some cases, the protein comprises an antibody or enzyme. In some cases, the cargo molecule comprises antisense oligonucleotide. In some cases, the cargo molecule comprises morpholino oligomer. In some cases, the cargo molecule comprises one or more components of a gene editing system. In some cases, the gene editing system is selected from the group consisting of CRISPR/Cas, zinc finger nuclease, transcription, and activator-like effector nuclease (TALEN).

In some cases, the plurality of extracellular vesicles comprises exosomes, microvesicles, apoptotic bodies, or any combinations thereof. In preferred cases, the plurality of extracellular vesicles comprises exosomes. In some cases, the detergent is removed from the detergent-mixture solution using dialysis or ultra-centrifugation. In some cases, the detergent is removed from the detergent-mixture solution by contacting the detergent-mixture solution with a detergent-removal agent. In some cases, the detergent-removal agent comprises a nonpolar polymeric adsorbent. In some cases, the nonpolar polymeric adsorbent comprises a polystyrene bead. In some cases, the nonpolar polymeric adsorbent comprises a SM-2 resin.

In one aspect, the method for processing EVs further comprises obtaining the plurality of extracellular vesicles from a cell culture medium. In some cases, the cell culture medium is derived from growing a plurality of cultured cells. In some cases, the plurality of cultured cells comprises stem cells, human embryonic kidney 293 (HEK 293) cells, HEK 293T cells, or any combinations thereof. In some cases, the stem cells comprise keratinocyte stem cells. In some cases, the method for processing EVs further comprises purifying the plurality of extracellular vesicles by centrifugation.

In one aspect, the present disclosure provides a kit for processing extracellular vesicles and comprises the detergent and detergent-removal agent of the present disclosure. In some cases, the kit further comprises the cargo molecule.

In one aspect, the present disclosure provides a composition comprising a plurality of cargo-loaded extracellular vesicles of the present disclosure. In some cases, the composition further comprises a pharmaceutically acceptable excipient.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a flowchart of an exemplary exosome production and purification protocol that utilizes HEK293F or Keratinocyte conditioned cell medium.

FIGS. 2A-2C show the physiochemical characterization of keratinocyte derived exosomes. FIGS. 2A-2B show electron microscopy images of exosome samples for the confirmation of exosome morphology and size. FIG. 2C shows a graph of the concentration vs particle hydrodynamic size.

FIGS. 3A-3C show the physiochemical characterization of HEK293F derived exosomes. FIGS. 3A-3B show electron microscopy images of exosome samples for the confirmation of exosome morphology and size. FIG. 3C shows a graph of the concentration vs particle hydrodynamic size.

FIGS. 4A-4D show an exemplary exosome cargo loading process in the presence or absence of a detergent. FIG. 4A is a schematic diagram of the exosome loading process. FIG. 4B is an overview of the exosome cargo loading. FIG. 4C is a chemical structure of an exemplary detergent, Triton X-100. FIG. 4D is a chemical structure of an exemplary detergent, octaethylene glycol monododecyl ether (C12E8).

FIG. 5 shows a bar graph representing the loading efficiency of FITC-labeled Dextran. The loading efficiency of FITC-Dextran into exosome under condition with or without the addition of detergent (Triton X-100) was measured by using the nano-Flow Cytometry.

FIGS. 6A-6C shows a graph representing the solubilization and reconstruction of exosomes. FIG. 6A is a graph representing the solubilization of Keratinocyte derived EV with increasing concentration of Triton X-100. FIG. 6B is a graph representing the solubilization of HEK293F derived EV with increasing concentration of Triton X-100. FIG. 6C is an exosome solubilization curve with the addition of the increasing volume of Triton X-100 (dots, woB) and particle concentrations were measured by nano-Flow Cytometry (FIG. 6C). Exosome reconstruction with the addition of Bio-Beads and extraction of Triton X-100 (square, wB) are also reported herein.

FIG. 7 shows a bar graph representing the loading efficiency of FAM-labeled siRNA. The loading efficiency of FAM-siRNA into exosomes under different conditions (i.e., exosome loading by incubation, electroporation, or detergent-assisted method) was measured by using nano-Flow Cytometry.

FIGS. 8A-8B show bar graphs representing the loading efficiency of Cy5-labeled siRNA in the presence or absence of octaethylene glycol monododecyl ether (C12E8). FIG. 8A is bar graph representing the loading efficiency of Cy5-labeled siRNA in HEK293F derived EV. FIG. 8B is bar graph representing the loading efficiency of Cy5-labeled siRNA in Keratinocyte derived EV.

### DETAILED DESCRIPTION OF THE INVENTION

Disclosed here are purified extracellular vesicles (EVs) that are engineered to incorporate sufficient amount of one or more therapeutic agents or any molecules of interest and a method of using the EVs to deliver an effective amount of the therapeutics or any molecules of interest to a target site.

### Definitions

The term “about” and its grammatical equivalents in relation to a reference numerical value and its grammatical equivalents as used herein can include a range of values plus or minus 10% from that value, such as a range of values plus or minus 10%, 9%, 8%, 7%, 6%, 5%, 4%, 3%, 2%, or 1% from that value. For example, the amount “about 10” includes amounts from 9 to 11. Unless otherwise indicated, some embodiments herein contemplate numerical ranges. When a numerical range is provided, unless otherwise indicated, the range includes the range endpoints. Unless otherwise indicated, numerical ranges include all values and sub ranges therein as if explicitly written out.

The singular forms “a,” “an,” and “the” are used herein to include plural references unless the context clearly dictates otherwise. Accordingly, unless the contrary is indicated, the numerical parameters set forth in this application are approximations that may vary depending upon the desired properties sought to be obtained by the present invention.

Unless otherwise indicated, open terms, for example “contain,” “containing,” “include,” “including,” and the like mean comprising.

The term “agent”, “active pharmaceutical ingredient (API)”, “therapeutics”, “therapeutic agent”, and “drug” are interchangeably used herein and comprise agents with pharmacological effects inducing a biological or medical response in an animal or human tissue or cell system desired by the researcher, veterinary, general practitioner or other physician, comprising changing biological system at molecular level (e.g., acting as inhibitors, activators, or modulators of proteins), the palliation or the symptoms or the disease or disorder treated; said agents can be chemical compounds, biological molecules with therapeutic activity (e.g., siRNAs, miRNAs, anti-miRNAs, shRNAs, etc., antibodies, antibody fragments recognizing specific epitopes), anti-tumor drugs, or radiotherapy drugs.

The term “biological sample” refers to any sample that is obtained from or otherwise derived from a biological entity such as an animal. Examples of biological samples include cells, tissues, organoids samples obtained from in vitro cell or tissue cultures or from in vivo. Non-limiting particular examples of biological samples include cytology samples, tissue samples, biological fluids, blood, urine, pre-ejaculate, nipple aspirates, semen, milk, sputum, mucus, pleural fluid, pelvic fluid, synovial fluid, ascites fluid, body cavity washes, eye brushings, skin scrapings, a buccal swab, a vaginal swab, a pap smear, a rectal swab, an aspirate, a needle biopsy, a section of tissue obtained for example by surgery or autopsy, plasma, serum, spinal fluid, lymph fluid, sweat, tears, saliva, tumors, or any suitable samples from biological entity thereof.

The term “cargo molecule” refers to any molecules or compounds that are or to be incorporated, capsulated, fused, or injected into a molecule transferring cargo (e.g., vesicles, exosomes, etc.) and may be chemical or biological molecules with or without therapeutic activity.

The terms “detergent” and “surfactant” are used interchangeably herein and are used to describe a compound that comprises both lipophilic and hydrophilic segments so that when added to water or solvents it reduces the surface tension of the system. The term “non-ionic detergent” means a detergent molecule that contains an uncharged, hydrophilic head group(s).

The term “extracellular vesicles” shall be understood with the meaning commonly known in the art and refers to vesicles containing membrane-coated cytoplasmic portions that are released from cells in the microenvironment. These vesicles represent a heterogeneous population comprising a plurality of types of vesicles, including “exosomes” and microvesicles, or apoptotic bodies, which can be told apart based on size, antigen composition and secretion modes. The terms “therapeutic delivery vesicle” and “therapeutic cargo” shall be understood to relate to any type of vesicle that is, for instance, obtainable from a cell, for instance a microvesicle (any vesicle shedded from the plasma membrane of a cell), an exosome (any vesicle derived from the endo-lysosomal pathway), an apoptotic body (from apoptotic cells), a microparticle (which may be derived from e.g., platelets), an ectosome (derivable from e.g., neutrophiles and monocytes in serum), prostatosome (obtainable from prostate cancer cells), cardiosomes (derivable from cardiac cells), etc. Furthermore, the terms “cargo molecule delivering vesicle” and “delivery vesicle” shall also be understood to potentially also relate to lipoprotein particles, such as LDL, VLDL, HDL and chylomicrons, as well as liposomes, lipid-like particles, lipidoids, etc. Essentially, the present disclosure may relate to any type of lipid-based structure (vesicular or with any other type of suitable morphology) that can act as a delivery or transport vehicle for cargo molecules.

The term “loading” or “loading extracellular vesicles” is understood in the present disclosure as an activity or status to result that the vesicles comprise one or more molecules of interest normally not present therein inside, within, and/or on their membrane surface of the vesicles.

The terms “purified” or “isolated” are used interchangeably and are intended to mean having been removed from its natural environment. The terms purified or isolated does not require absolute purity or isolation; rather, it is intended as a relative term.

Unless defined otherwise, all technical and scientific terms used herein have the same meaning as commonly understood by one of the ordinary skill in the art to which this invention belongs. Although any methods and materials similar or equivalent to those described herein can be used in the practice or testing of the formulations or unit doses herein, some methods and materials are now described. Unless mentioned otherwise, the techniques employed or contemplated herein are standard methodologies. The materials, methods and examples are illustrative only and not limiting.

### Methods for Manufacturing Extracellular Vesicles (EVs)

Disclosed herein are EVs that comprise at least one cargo molecule. Also disclosed herein is a method of manufacturing the EVs for loading a sufficient amount of one or more cargo molecules so that an appropriate amount of the one or more cargo molecules is delivered to or within a target cell or tissue of interest.

In one aspect, the disclosure provides a method for processing extracellular vesicles (EVs), in which the method comprises the steps of contacting a biological sample comprising a plurality of extracellular vesicles with a cargo molecule and a detergent to form a detergent-mixture solution; and contacting the detergent-mixture solution with a detergent-removal agent to obtain a plurality of cargo-loaded extracellular vesicles.

The cargo molecules of the present disclosure may be any compounds, molecules, or peptides with or without biological activities. In some cases, the cargo molecule comprises an active pharmaceutical ingredient (API). In some cases, the cargo molecule may be therapeutic agents. The therapeutic agents according to the present description can be chemical compounds, such as anti-tumor drugs, radiotherapy drugs, antibiotics, or biological molecules with therapeutic activity (e.g., siRNAs, miRNAs, anti-miRNAs, shRNAs, etc., antibodies, antibody fragments, peptides, etc.). In some cases, the cargo molecule comprises a polypeptide, protein, lipid, nucleic acid, metabolite, or any combinations thereof. In some cases, the nucleic acid is selected from the group consisting of a DNA, mRNA, small interfering RNA (siRNA), microRNA (mi-RNA), and long non-coding RNA (IncRNA). Other exemplary cargo molecules include, but are not limited to, a protein, a carbohydrate, a lipid, a small molecule therapeutics, a toxin, an antibody, a recombinant protein, a viral vector, a vaccine, an antisense oligonucleotide, a gene editing system (e.g., CRISPR/Cas9 system), or any combination thereof.

In some cases, the one or more cargo molecules have a final concentration of about 0.1 μM to about 300 μM. In some cases, the one or more cargo molecules have a final concentration of at least about 0.1 μM to about 1 μM, about 0.1 μM to about 10 μM, about 0.1 μM to about 20 μM, about 0.1 μM to about 50 μM, about 0.1 μM to about 100 μM, about 0.1 μM to about 150 μM, about 0.1 μM to about 200 μM, about 0.1 μM to about 300 μM, about 1 μM to about 10 μM, about 1 μM to about 20 μM, about 1 μM to about 50 μM, about 1 μM to about 100 μM, about 1 μM to about 150 μM, about 1 μM to about 200 μM, about 1 μM to about 300 μM, about 10 μM to about 20 μM, about 10 μM to about 50 M, about 10 μM to about 100 μM, about 10 μM to about 150 μM, about 10 μM to about 200 μM, about 10 μM to about 300 μM, about 20 μM to about 50 μM, about 20 μM to about 100 μM, about 20 μM to about 150 μM, about 20 μM to about 200 μM, about 20 μM to about 300 μM, about 50 μM to about 100 μM, about 50 μM to about 150 μM, about 50 μM to about 200 μM, about 50 μM to about 300 μM, about 100 μM to about 150 μM, about 100 μM to about 200 μM, about 100 μM to about 300 μM, about 150 μM to about 200 μM, about 150 μM to about 300 μM, or about 200 μM to about 300 μM. In some cases, the one or more cargo molecules have a total concentration of at least about 0.1 μM, about 1 μM, about 10 μM, about 20 μM, about 50 μM, about 100 μM, about 150 μM, about 200 μM, or about 300 μM.

In some cases, the biological samples comprising a plurality of EVs form a detergent-mixture solution by contacting a detergent. In some cases, the detergent comprises a surfactant. In these cases, the surfactant comprises a hydrophilic group and a hydrophobic group. In some cases, the hydrophilic group of the surfactant comprises a polyethylene oxide chain. In some cases, the surfactant is a non-ionic surfactant. Exemplary non-ionic detergents (or surfactants) in the detergent-mixture solution include, but are not limited to, BigCHAP(N,N-Bis[3-(D-glucona-mido)propyl]cholamide), Bis(polyethylene glycol bis[imidazoyl carbonyl]), Brij® 30 (Polyoxyethylene 4 lauryl ether) Brij®35 (Polyoxyethylene 23 lauryl ether), Brij®52 (Polyoxyethylene 2 cetyl ether), Brij®56 (Polyoxyethylene 10 cetyl ether), Brij® 58 (Polyoxyethylene 20 cetyl ether), Brij®72 (Polyoxyethylene 2 stearyl ether), Brij®76 (Polyoxyethylene 10 stearyl ether), Brij®78 (Polyoxyethylene 20 stearyl ether), Brij®92 (Polyoxyethylene 2 oleyl ether), Brij®97 (Polyoxyethylene 10 oleyl ether), Brij®98 (Polyoxyethylene 20 oleyl ether), Brij®700 (Polyoxyethylene 100 stearyl ether), Cremophor®EL (castor oil/ethylene oxide polyether), Decaethylene glycol monododecyl ether, octanoyl-N-methylglucamide (MECA-8), decanoyl-N-methylglucamide (MECA-10), n-octylglucoside, ndodecylglucoside, isotridecyl-poly(ethyleneglycolether)n, N-Decanoyl-N-methylglucamine, n-Decyl-α.-D-glucopyranoside, Decyl-β-D-maltopyranoside, n-Dodecanoyl-N-methylglucamide, n-Dodecyl-α.-D-maltoside, n-Dodecyl-β-D-maltoside, Heptaethylene glycol monodecyl ether, Heptaethylene glycol monotetradecyl ether, n-Hexadecyl-β-D-maltoside, Hexaethylene glycol monododecyl ether, Hexaethylene glycol monohexadecyl ether, Hexaethylene glycol monooctadecyl ether, Hexaethylene glycol monotetradecyl Igepal® CA-630 (Octylphenyl-polyethylene glycol), Igepal® CA210 (polyoxyethylene(2) isooctylphenyl ether), Igepal® CA-520 (polyoxyethylene(5) isooctylphenyl ether), Igepal®' CO630 (polyoxyethylene(9)nonylphenyl ether), Igepal®CO-720 (polyoxyethylene(12) nonylphenyl ether), Igepal® CO-890 (polyoxyethylene(40)nonylphenyl ether), Igepal® CO-990 (polyoxyethylene(100) nonylphenyl ether), Igepal® DM-970 (polyoxyethylene(150) dinonylphenyl ether), Methyl-6-O—(N-heptylcarbamoyl-)-. alpha.-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-.beta.-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin, Span® 20 (Sorbitan monolaurate), Span® 40 (Sorbitan monopalmitate), Span® 60 (Sorbitan monostearate), Span® 65 (Sorbitan tristearate), Span® 80 (Sorbitan monooleate), Span® 85 (Sorbitan trioleate), Tergitol in any form (including Types 15-S-5, 15-S-7, 15-S-9, 15-S-12, 15-S-30, NP-4, NP-7, NP-9, NP-10, NP-40, NPX (Imbentin-N/63), TMN-3 (Polyethylene glycol trimethylnonyl ether), TMN-6 (Polyethylene glycol trimethylnonyl ether), TMN-10 (Polyethylene glycol trimethylnonyl ether), MIN FOAM 1×, and MIN FOAM 2×), Tetradecyl-.beta.-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton® CF-21, Triton® CF-32, Triton@ DF-12, Triton® DF-16, Triton® GR5M, Triton® N-1 01 (Polyoxyethylene branched nonylphenyl ether), Triton® QS-15, Triton® QS-44, Triton® RW-75 (Polyethylene glycol 260 mono (hexadecyl/octadecyl) ether and 1-Octadecanol), Triton® X-100 (Polyethylene glycol tertoctylphenyl ether), Triton® X-102, Triton® X-15, Triton® X-151, Triton® X-200, Triton® X-207, Triton® X-114, Triton® X165, Triton® X-305, Triton® X-405 (polyoxyethylene (40) isooctylphenyl ether), Triton® X-405 reduced (polyoxyethylene (40) isooctylcyclohexyl ether), Triton® X-45 (Polyethylene glycol 4-tert-octylphenyl ether), Triton® X-705-70, TWEEN® in any form (including TWEEN® 20 (Polyoxyethylenesorbitan monolaurate), TWEEN® 21 (Polyoxyethylenesorbitan monolaurate), TWEEN® 40 (polyoxyethylene(20) sorbitan monopalmitate), TWEEN® 60 (Polyethylene glycol sorbitan monostearate), TWEEN® 61 (Polyethylene glycol sorbitan monostearate), TWEEN® 65 (Polyoxyethylenesorbitan Tristearate), TWEEN® 80 (Polyoxyethylenesorbitan monooleate), TWEEN® 81 (Polyoxyethylenesorbitan monooleate), and TWEEN® 85 (polyoxyethylene(20) sorbitan trioleate)), Tyloxapol (4-(1,1,3,3-tetramethylbutyl)phenol polymer with formaldehyde and oxirane), and n-Undecyl β-D-glucopyranoside. In some cases, the detergent is 2-[4-(2,4,4-trimethylpentan-2-yl)phenoxy] ethanol. In some cases, the detergent is dodecyloctaglycol or octaethylene glycol monododecyl ether. In some cases, the detergent is octaethylene glycol monododecyl ether.

Alternatively, additional detergent may be added to the detergent-mixture solution comprising a non-ionic detergent. The additional detergent may be an anionic detergent. Exemplary anionic detergents include Chenodeoxycholic acid, Cholic acid, Dehydrocholic acid, Deoxycholic acid, Digitonin, Digitoxigenin, N,N-Dimethyldodecylamine N-oxide, Sodium docusate, Sodium glycochenodeoxycholate, Glycocholic acid, Glycodeoxycholic acid, Glycolithocholic acid 3-sulfate disodium salt, Glycolithocholic acid ethyl ester, N-Lauroylsarcosine, Lithium dodecyl sulfate, Lugol (Iodine Potassium Iodide), Niaproof (2-Ethylhexyl sulfate sodium salt), Niaproof 4 (7-Ethyl-2-methyl-4-undecyl sulfate sodium salt), optionally substituted alkylsulfonate salts (including salts of 1-butanesulfonate, pentanesulfonate, hexanesulfonate, 1-Octanesulfonate, 1-decanesulfonate, 1-dodecanesulfonate, 1-heptanesulfonate, 1-heptanesulfonate, 1-nonanesulfonate, 1-propanesulfonate, and 2-bromoethanesulfonate, especially the sodium salts), Sodium cholate, Sodium deoxycholate, optionally substituted Sodium dodecyl sulfate, Sodium octyl sulfate, Sodium taurocholate, Sodium taurochenodeoxycholate, Sodium taurohyodeoxycholate, Taurolithocholic acid 3-sulfate disodium salt, Tauroursodeoxycholic acid sodium salt, Trizma® dodecyl sulfate, Ursodeoxycholic acid. An anionic detergent can be provided in acid or salt form, or a combination of the two. Exemplary cationic detergents include Alkyltrimethylammonium bromide, Benzalkonium chloride, Benzyldimethylhexadecylammonium chloride, Benzyldimethyltetradecylammonium chloride, Benzyldodecyldimethylammonium bromide, Benzyltrimethylamnonium tetrachloroiodate, Dimethyldioctadecylammonium bromide, Dodecylethyldimethylammonium bromide, Dodecyltrimethylammonium bromide, Ethylhexadecyldimethylammonium bromide, Girard's reagent T, Hexadecyltrimethylammonium bromide, N,N′,N′-Polyoxyethylene(10)-N-tallow-1,3-diaminopropane, Thonzonium bromide, and Trimethyl(tetradecyl)aminonium bromide. Exemplary zwitterionic detergents include CHAPS (3-{(3-cholamidopropyl)-dimethylammonio}-1-propane-sulfonate), CHAPSO (3-{(3-cholamidopropyl)dimethylammonio}-2-hydroxy-1-propane-sulfonate), 3-(Decyldimethylammonio)propanesulfonate, 3-(Dodecyldimethylammonio)propanesulfonate, 3-(N,N-Dimethylmyristylammonio)propanesulfonate, 3-(N,N-Dimethyloctadecylammonio)propanesulfonate, 3-(N,N-Dimethyloctylamm-onio)propanesulfonate, and 3-(N,N-Dimethylpalmitylammonio)propanesulfonate. Additional exemplary detergents include, but are not limited to, octylglucoside, octylphenoxy poly(ethyleneoxy)ethanol (nonidet® P-40), and 1,2-Distearoyl-sn-glycerol-3-phosphocholine (DSPC). Combinations of two or more detergents, and combinations of one or more detergents and one or more other lipid compounds also are contemplated.

In some cases, the one or more detergents produce low or no toxicity in cells. In these cases, the detergent is a nonionic detergent. In such cases, the detergent is Polyethylene glycol p-(1,1,3,3-tetramethylbutyl)-phenyl ether (Triton X-100®). In some cases, the detergent is octaethylene glycol monododecyl ether (OEG). In some cases, the EVs comprise polyethylene glycol p-(1,1,3,3-tetramethylbutyl)-phenyl ether at a final concentration of about 0.03 mM to about 4 mM, about 0.04 mM to about 3 mM, about 0.05 mM to about 2.5 mM, about 0.06 mM to about 2.2 mM, about 0.08 mM to about 2 mM, about 0.1 mM to about 1.8 mM, about 0.2 mM to about 1.5 mM or any concentration in between thereof.

In some cases, the biological samples that comprise a plurality of EVs may form a detergent-mixture solution having a final detergent concentration of about 0.005% v/v to about 10% v/v, about 0.01% v/v to about 9.8% v/v, about 0.02% v/v to about 9.6% v/v, about 0.04% v/v to about 9.4% v/v, about 0.06% v/v to about 9.2% v/v, about 0.08% v/v to about 9.0%, about 0.1% v/v to about 8.0% v/v, about 0.1% v/v to about 7.0% v/v, about 0.1% v/v to about 6.0% v/v, about 0.1% v/v to about 5.0% v/v, about 0.2% v/v to about 4.0% v/v, about 0.4% v/v, about 0.5% v/v, about 0.6% v/v, about 0.8% v/v, about 1.0%, about 1.2%, about 1.4%, about 1.6%, about 1.8%, about 2.0%, about 2.2%, %, about 2.4%, about 2.6%, about 2.8%, about 3.0%, about 3.2%, about 3.4%, %, about 3.6%, about 3.8%, about 4.0%, about 4.2%, about 4.4%, about 4.6%, about 4.8%, about 5.0% or any concentrations in between.

In some cases, the biological sample comprising extracellular vesicles may contact the cargo molecule and the detergent by adding the cargo molecule and the detergent simultaneously. In some cases, the biological sample comprising extracellular vesicles may contact the cargo molecule and the detergent by adding the cargo molecule and the detergent sequentially. In some cases, the method comprises a step of contacting the biological sample comprising a plurality of EVs with the cargo molecule and the detergent by adding the cargo molecule prior to adding the detergent. In other cases, the method comprises a step of contacting the biological sample comprising a plurality of EVs with the cargo molecule and the detergent by adding the detergent prior to adding the cargo molecule.

In some cases, more than one non-ionic detergent may be included in a detergent-mixture solution to disrupt lipid-based membrane structure of a plurality of types of EVs, such as virosomes or fusogenic liposomes. In these cases, preferred non-ionic detergent may be any non-ionic detergent that has a hydrophilic region and a hydrophobic and/or lipophilic region so that the lipophilic region interacts with the lipid components within the lipid-membrane based structures. In some cases, the EVs may be assembled in vitro in a cell-free system with part of their lipid membrane sourcing from purified viral membrane components. Exemplary viruses for the assembly of EVs include, but are not limited to, influenza virus, hepatitis B virus, human immunodeficiency virus, Newcastle disease virus, and Sendai virus.

In some cases, the detergent-mixture solution is contacted with a detergent-removal agent to obtain a plurality of cargo-loaded EVs. In such cases, the detergent-removal agent comprises a nonpolar polymeric adsorbent. In some cases, the nonpolar polymeric adsorbent comprises a polystyrene bead. Some exemplary nonpolar polymeric adsorbent beads include, but are not limited to, HP-20 (Mitsubishi chemical), SP-825 (Mitsubishi), Amberlite XAD-2 and XAD-4 (Rohm and the manufacturing of Haas) and Duolite S-861, S-862 (Sumitomo chemical), and SM-2 (Bio-Rad laboratories). In some cases, the nonpolar polymeric adsorbent may be SM-2 resin.

In some cases, the detergent-removal agent may be incorporated into a purification column (e.g., chromatography column) for a larger volume EVs loading process. In these cases, the purification column may be packed with an appropriate amount (for example, 10 to 100% w/w. 20 to 100% w/w, 30 to 100% w/w, 40 to 100% w/w, 50 to 100% w/w, 60 to 100% w/w, 70 to 100% w/w, 80 to 100% w/w, etc.) of the detergent-removal agent. In some cases, the purification column is packed with SM-2 resins. In some cases, the purification column is packed with any detergent-removal agent listed above. The prepared purification column may then be used to remove the excess detergent from the cargo-loaded EVs after the detergent-assisted loading process.

In some cases, the detergent may be removed by utilizing other methods. For example, the use of dialysis or ultracentrifugation (e.g., discontinuous sucrose-gradient centrifugation) may be contemplated for removing the detergent and/or other impurities following the loading of EVs.

In another aspect, the method for processing EVs further comprises obtaining the plurality of EVs from a cell culture medium. In some cases, the cell culture medium is derived from growing a plurality of cultured cells. In some cases, the plurality of cultured cells comprises stem cells, human embryonic kidney 293 (HEK 293) cells, HEK 293T cells, or any combinations thereof. In some cases, the stem cells comprise keratinocyte stem cells.

In some cases, the biological sample may be purified cell culture medium. In other cases, the biological sample may be unpurified cell culture medium. The biological sample comprising a plurality of vesicles may be filtered through a filter with a suitable mesh or pore size (e.g., nylon mesh cell strainers). The filter may have the pore size of 50 n m to 100 μM. In some cases, the filter may have the pore size of 80 nm to 90 μM. In some cases, the filter may have the pore size of 100 nm to 80 μM. In some cases, the filter may have the pore size of about 200 nm to about 70 μM, about 400 nm to about 60 μM, about 600 nm to about 50 μM, about 800 nm to about 40 μM, or about 1 μM to about 20 μM. In some cases, the method for processing EVs further comprises purifying the plurality of extracellular vesicles by centrifugation.

In some cases, the plurality of EVs may be purified or isolated from cells, cell culture medium, or tissues as described in Example 1. In some cases, the plurality of EVs may be purified or isolated prior to contacting a detergent or cargo molecule. In some cases, the plurality of EVs may be purified or isolated after contacting a detergent-removal agent. In some cases, the EVs may be purified or isolated as the plurality of cargo-loaded EVs.

In some cases, the EVs have an average diameter length of at least about 80 nm. In some cases, the EVs have an average diameter length of at least about 180 nm. In some cases, the EVs have an average diameter length of at least about 80 nm to about 180 nm, about 85 nm to about 175 nm, about 90 nm to about 160 nm, about 92 nm to about 150 nm, about 96 nm to about 140 nm, about 98 nm to about 130 nm, about 100 nm to about 120 nm, about 102 nm to about 112 nm, or about 105 nm to about 110 nm. The size of the EVs may change following loading of the cargo molecules. In other cases, the size of the EVs may remain the same after loading.

In another aspect, the present disclosure also provides a kit for processing EVs, which comprises the detergent and detergent-removal agent of the present disclosure as described above. In some cases, the kit further comprises the cargo molecule of the present disclosure.

In some cases, the method of the present disclosure provides at least about 1% to about 40% of active pharmaceutical ingredient (API) loading efficiency, for example, at least about 5% to about 40%, at least about 10% to about 40%, or at least about 15% to about 40%, about 1%, about 2%, about 4%, about 6%, about 8%, about 10%, about 12%, about 14%, about 16%, about 18%, about 20%, about 22%, about 24%, about 26%, about 28%, about 30%, about 32%, about 34%, about 36%, about 38%, or about 40%, including any values or ranges therebetween. In some cases, the method of the present disclosure provides about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% API loading efficiency onto the EVs, including any values or ranges therebetween. In some cases, the method provides at least about 20% or about 25% of API loading efficiency (%). In some cases, the method provides about 30% or about 40% of API loading efficiency (%). In some cases, the method provides about 30-50% of API loading efficiency (%).

### Method of using EVs

A method of using EVs of the present disclosure for treating a patient suffering from chronic and recurrent diseases by administering an effective amount of the EVs to the patient is disclosed herein. The chronic and recurrent diseases may be diabetes, infection, protein deficiencies, or immunological disorders.

In the therapeutic method of the present disclosure, the EVs may be administered to the patient via intravenous, intra-arterial, intranasal, or topical administration route. The effective dosage could be evaluated by the attending physician on an empirical basis or set by in vivo or in vitro evaluation for each pathology.

### EXAMPLES

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1. Method of Isolating and Purifying Exosomes from Mammalian Cells

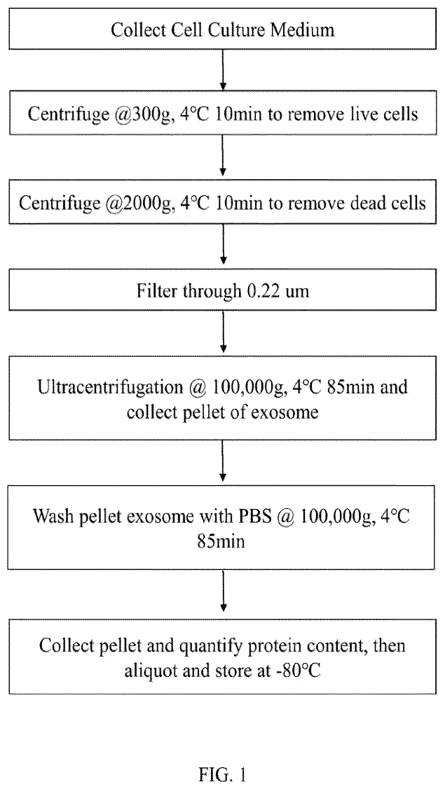

Referring to FIG. 1, the differential centrifugation protocol that was used for the isolation and purification of exosomes from various types of mammalian cells is outlined in a flow chart.

Briefly, exosomes were produced from conditioned medium derived from growing several types of cultured cells, including Keratinocyte stem cells and HEK 293T. 293T cells were cultured in DMEM supplemented with 10% fetal bovine serum and maintained in a humid incubator with 5% CO₂ at 37° C. The keratinocyte stem cells were cultured in EpiZero culture medium. For purification of exosomes, the above mentioned fetal bovine serum was replaced by a type of exosomes-depleted serum. Exosomes from the supernatant of keratinocyte culture medium were directly subjected to the purification process without replacing the serum to an exosome-depleted serum.

Keratinocyte stem cells or 293T cells were seeded in 175-cm² flasks at a cell density of 4×10⁶ or 3×10⁶ cells respectively for 5 days. Cell culture supernatant was harvested into 50 mL centrifuge tubes and immediately subjected to a two-step centrifugation at 4° C., 300×g for 10 min, 2000×g for 10 min, and filtered through a 0.22-μm membrane to remove cells, cell fragments, shedding vesicles and other debris. At each step, the supernatant was carefully transferred to new tubes. The exosome fraction was concentrated by ultracentrifugation in a 38.5 mL Polypropylene centrifuge tubes in a SW32Ti rotor at 100,000×g for 85 min at 4° C. The pellet was washed with 12.5 mL of PBS and followed by a second ultracentrifugation at 100,000×g for 85 min at 4° C. Ultimately, the supernatant was discarded and EVs were resuspended in 100 μL PBS. Exosomes collected from 293T cell culture supernatant were subjected to size exclusion chromatography (SEC) with SEC columns. The concentration of the collected exosomes was quantified by measuring the protein concentration through a BCA Protein Quantification Assay.

The physicochemical properties of the isolated exosomes were further characterized. To observe the morphology of the isolated exosomes, the exosome-containing samples and transmission electron microscopy (TEM) grids (copper) for electron microscopy were prepared at room temperature. 10 μL aliquots of extracellular vesicle samples were dropped onto the grids and then air dried for 10 minutes. The extra liquids were dried with a filter paper. The grids were then washed with 10 μL PBS and quickly drying with filter paper. For the negative staining, 10 μL of 2% uranyl acetate was dropped onto the sample grids and timed staining for 1-3 minutes, then the extra liquid was removed with a filter paper. Samples were allowed to air-dry for 10 minutes before imaging. TEM images were examined in a JEOL 1200EX transmission electron microscope at 100 kV and images were obtained with a CCD camera.

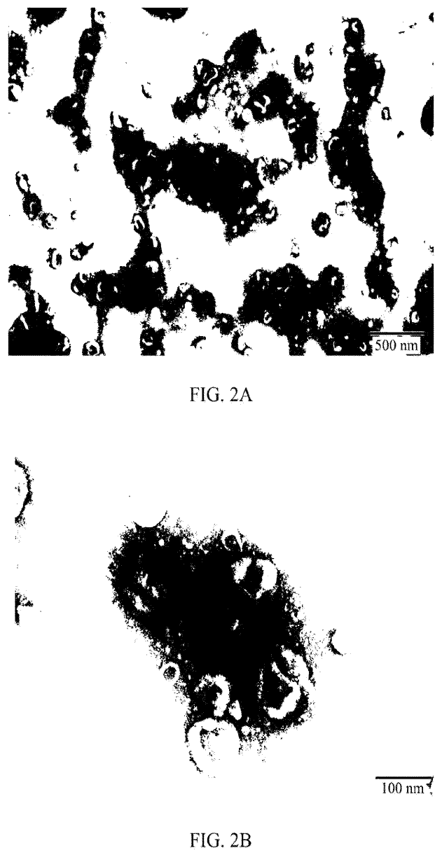

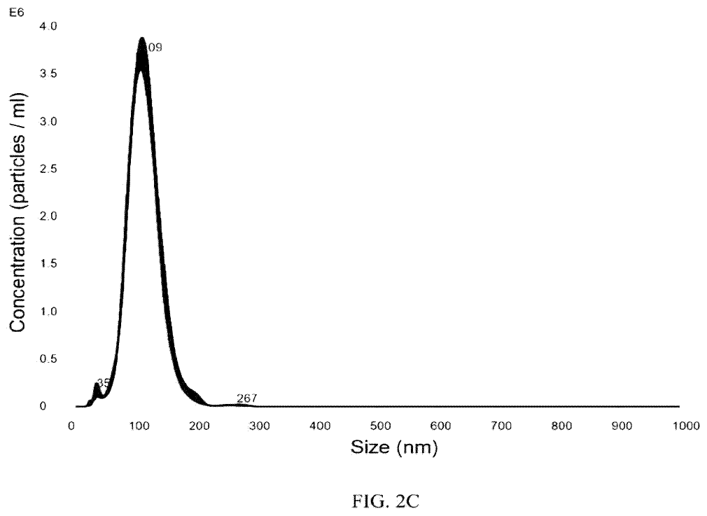

As shown in FIGS. 2A and 2B, the isolated exosomes exhibited a “saucer-like” morphology, with an average diameter of around 100 nm. The main peak of the size distribution at 106 nm was further confirmed by nanoparticle tracking analysis, where the effective hydrodynamic diameter of exosomes in aqueous environment was measured and calculated by monitoring the Brownian motion of the particles in solution (FIG. 2C).

### Example 2. Method for Solubilizing and Reconstituting of Exosomes

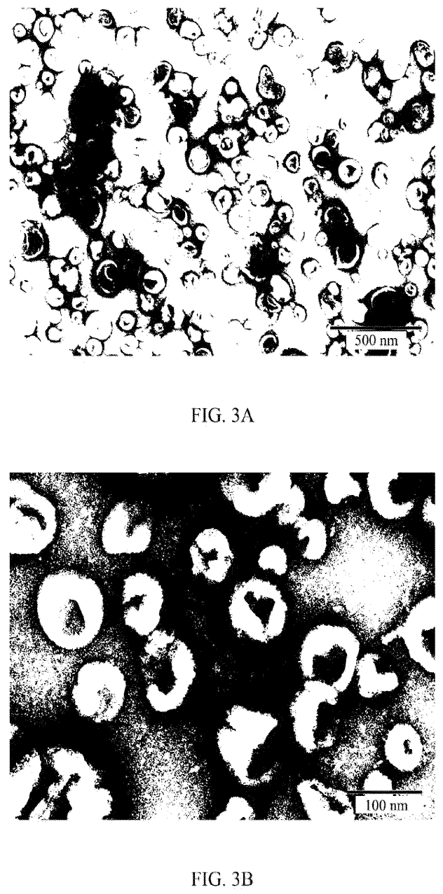

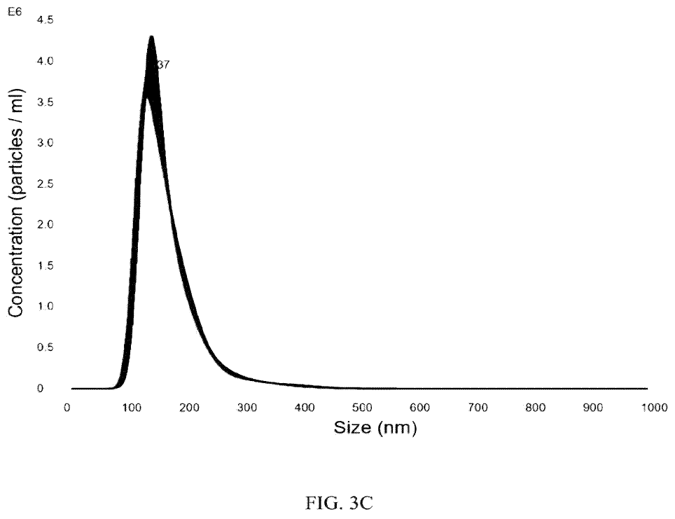

The scheme of loading potential therapeutic cargo molecules into exosome is illustrated in FIG. 3A, representing the membraned structure of exosome being made permeable by the addition of a non-ionic detergent (loading helper). The cargo molecules (potentially could be small chemical drug molecules, miRNAs, siRNAs, IncRNAs, mRNAs, proteins/peptides or other cargo molecules) were loaded onto the exosomes by incubating with exosomes in the presence of a loading helper. Eventually, the loading helper was extracted from the loading environment by adding the Bio-Beads for a hydrophobic interaction, leaving behind exosomes loaded with cargo molecules in the reconstructed membraned structure, as shown in FIG. 3B. In order to find a preferred loading condition, exosomes were first solubilized by adding increasing amounts of Triton X-100. In brief, 1-10 μg of exosomes were dispersed in Triton X-100 containing PBS solution at a volume concentration of 0, 0.1, 0.2, 0.4, 0.5, 0.6, 0.8, 1.0, 5.0% v/v with 200 μL of total volume (corresponding to a range of 0 to 2 mM). The solution was incubated at 4° C. or 37° C. for an hour, then samples were incubated at 4° C. before being characterized by nFCM or NTA. The reconstitution of exosomes was achieved by first solubilizing exosomes at varying Triton X-100 conditions, followed by the addition of Bio-Beads for extracting Triton X-100 from the sample at 4° C. Samples after the reconstitution were analyzed by Nano-Flow Cytometry (nFCM) or Nanoparticle tracking analysis (NTA).

For nFCM analysis, the exosome samples were prepared for the Apogee A60 Micro Plus Flow Cytometer uptake, in which the analyzing cytometer was specially developed for analysis of nanoparticles. Three spatially separated lasers (488 nm—Position C, 638 nm—Position B and 405 nm—Position A) were in the A60-Micro-Plus machine with 6 fluorescence color detectors (445/50, 530/40, 575/30, LWP650, 676/36, LWP750) and 3 light scatter detectors (small angle light scatter (SALS), multi angle light scatter (MALS) and low angle light scatter (LALS). In some cases, before or during the sample measurements, two commercially available mixes of beads were used as the control and calibration standards. Exosome samples were diluted in PBS to within the operational range of the equipment (maximum of 3,000 events/second). To analyze the size and concentration, samples were processed at a flow rate of 1.50 μL/min using a 405 nm-MALS threshold of 15 (instrument arbitrary number). The 405 nm-MALS PMT was monitored and always maintained below 0.5, which is considered as an indicator of the background noise. PBS was also measured in between samples as internal controls and analyzed for particle concentrations as an indicator of background signal levels.

For NTA analysis, exosome particle size and concentration were assessed using Brownian motion NanoSight NS300 system. Exosome samples were diluted at room temperature in 1 mL PBS within the operational range and pumped into the instrument at fixed pump speed of 15 (instrument arbitrary unit). The motion of particles was monitored, and the recorded videos were analyzed by using the NTA software.

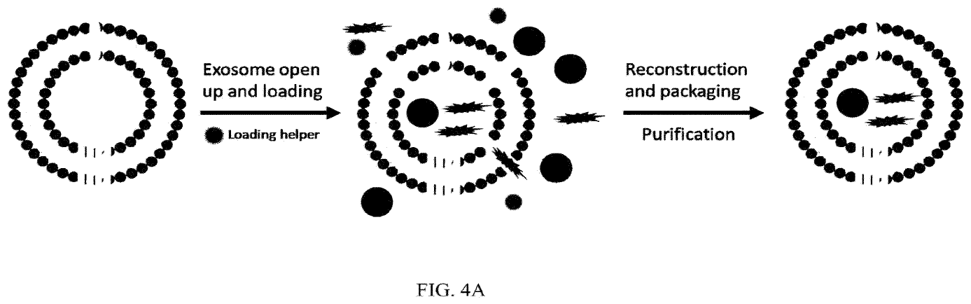

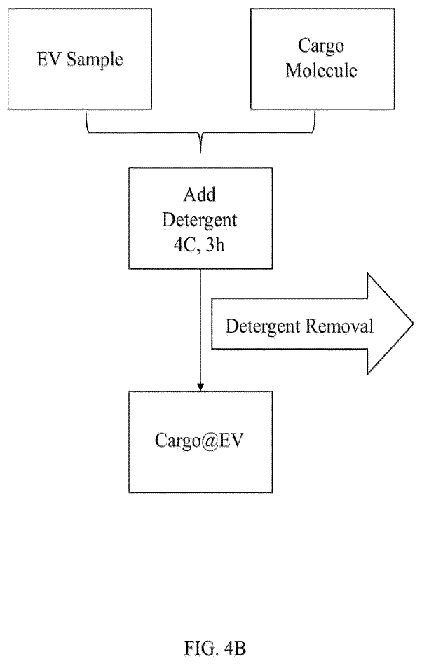

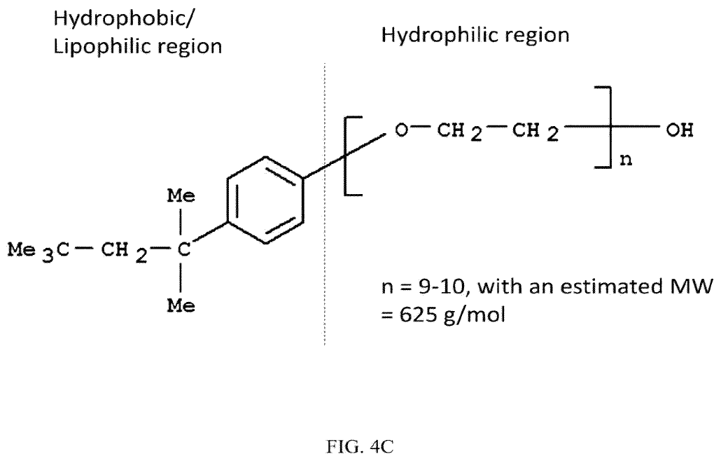

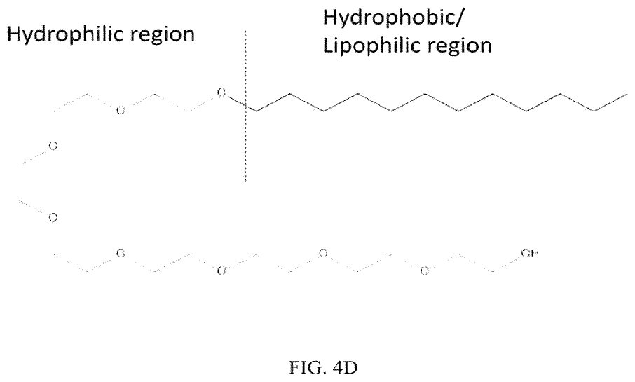

The analyzed particles number are plotted as shown in FIG. 3C. The particle number was rescaled based on the particle number of untreated samples, which is set to 1.0. As shown in FIGS. 4A-4C, the exosomes were solubilized as the increasing amounts of Triton X-100 were added to the system. The exosomes were reconstructed in the presence of the Bio-beads, as shown by the elevation of the relative levels of particle number at a Triton X-100 concentration of 0.09 mM and 0.125 mM. Thus, the Triton X-100 concentration of 0.125 mM was used in the following loading experiments.

### Example 3. Method of Detergent-Assisted Exosome Loading of FITC-Dextran

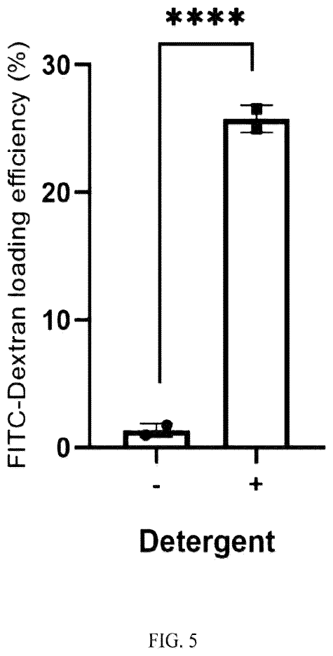

A loading solution of FITC-Dextran was prepared at a concentration of 8 nM in PBS. The hydrodynamic diameter of a FITC-Dextran (wt=40,000) was estimated around 4 nm, and FD40 was used as a macromolecule model for protein trafficking estimation. 4 μg of exosomes were dispersed in 100 μL FITC-Dextran loading solution with the addition of specific volume of Triton X-100 from the stock solution (2% v/v). An optimized loading condition was chosen from the solubilization and reconstruction experiment, and Bio-Beads were added in the Eppendorf tubes to extract Triton X-100 after loading. Samples were then separated from the Bio-Beads by low-speed centrifugation and stored in 4° C. before being analyzed by nFCM or NTA. The loading efficiency of FITC-Dextran was estimated by nano Flow cytometry where the ratio of EVs with green fluorescence signal was measured over the total events of EVs. The results are summarized in FIG. 5.

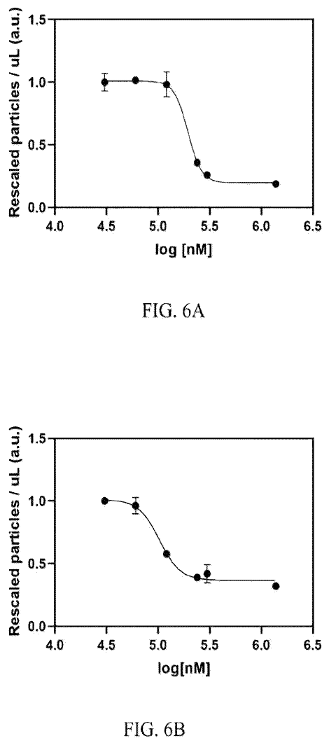

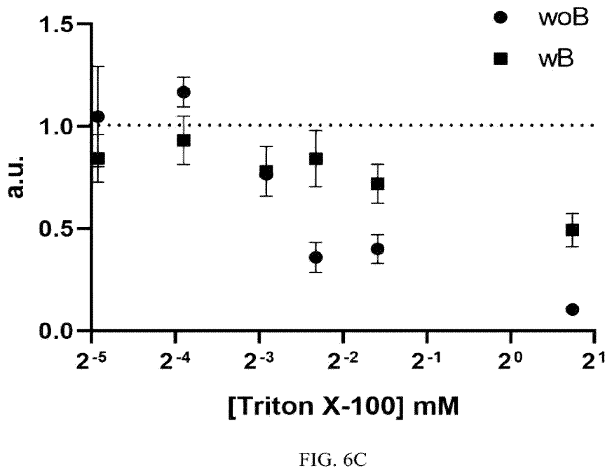

Referring to FIGS. 6A-6C, a detergent-assisted reconstruction loading process enabled 31% more of EVs being loaded with FITC-dextran as compared to the incubation process, indicating the capability of such method to load EVs with macromolecules with similar hydrodynamic size of around 4 nm. The solubilization of Keratinocyte derived EV with increasing concentration of 2-[4-(2,4,4-trimethylpentan-2-yl)phenoxy]ethanol (Triton X-100) and the solubilization of HEK293F cell derived EV with increasing concentration of Triton X-100 are shown in FIGS. 6A and 6B. As shown in FIG. 6C, exosome solubilization with increasing concentration of Triton X-100 was analyzed by using nano-Flow Cytometry.

### Example 4. Method of Loading Florescent Dye (FAM)-Sirna in Exosomes by Electroporation

The loading of FAM-siRNA in exosomes by electroporation was achieved by incubating 4 μg equivalent exosomes in 1×PBS and 20 pM of FAM-siRNA loading solution at 4° C. Then the electroporation was applied with a Bio-Rad electroporation instrument at 400V, 25 μF and pulse electroporation protocol known in the art, or with a Lonza 3D electroporation instrument with ED120 or ED137 electroporation protocol, also well known in the art. The loading efficiency of FAM-siRNA into exosomes was measured by nano flow cytometry similar to that of FITC-dextran, where effective loading could be observed by a shift of MALS signal of exosome into the green fluorescence signals.

### Example 5. Comparative Analysis of Detergent-Assisted and Electroporated Exosomes

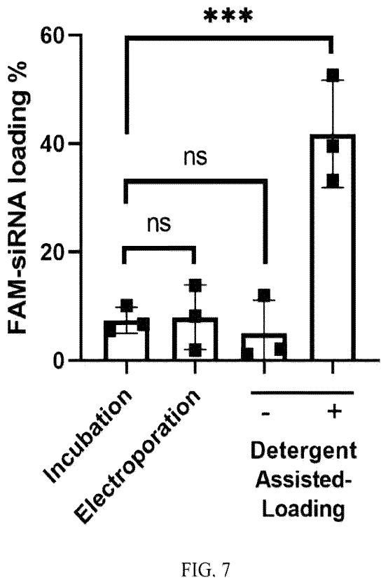

In a typical exosome loading experiment of siRNA, 1 μL of 20 pM FAM-siRNA was first dispersed in 100 μL OptiMEM, and then 4 μg equivalent exosome in PBS was added to the microcentrifuge tube at 4° C. for 10 min for equilibration. 0.8 μL of 2% (v/v) Triton X-100 was added to the system, and the whole loading system was incubated at 4° C. for 2 hours with gentle rotation. Then the system was diluted by addition of 900 μL of 1× PBS, and 10 Bio-Beads per microcentrifuge tube were added at 4° C. for 3 h incubation with gentle rotation. Another 10 Bio-Beads were added, and microcentrifuge tube was incubated at 4° C. rotation for an additional 1 hour up to overnight. The Bio-Beads were isolated from the sample by low-speed centrifugation, then the loaded exosomes were collected with or without further purification by SEC and subjected to nFCM or NTA analysis. The loading efficiency of detergent-assisted process was compared to that of incubation and electroporation process. Four different siRNA loading conditions were tested: i) a control, incubation in electroporation buffer, ii) electroporation in electroporation buffer, iii) another control, in detergent-assistant loading buffer in the absence of detergent, and iv) detergent-assistant loading in the presence of Triton X-100, and the results are depicted in FIG. 7. The effective loading of FAM-siRNA into exosomes was confirmed by nano flow cytometry, where positive green-fluorescent signal of FAM coincided with the scattering signal of exosome detected by MALS. The loading efficiency was quantified by nano flow cytometry in percentage of green fluorescence positive exosomal events, overall exosome events measured, and the mean fluorescent intensity shift when compared to non-loading controls. As shown in FIG. 7, the loading efficiency of FAM-siRNA via the detergent-assisted reconstruction method was more than 3-fold higher than the loading by using the incubation or electroporation method.

### Example 6. Method of Detergent-Assisted Exosome Loading of Cy5-siRNA

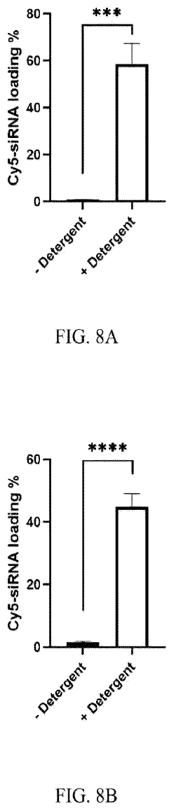

This study was designed to evaluate loading efficiency of Cy5-siRNA in the presence or absence of octaethylene glycol monododecyl ether (C12E8). Initially, the HEK293F- or keratinocyte-derived exosomes were dispersed in Cy5-siRNA loading solution with the addition of specific volume of C12E8 from the stock solution. An optimized loading condition was chosen from the solubilization and reconstruction experiment. The loading efficiency was estimated by nano Flow cytometry where the ratio of EVs with green fluorescence signal was measured over the total events of EVs. The results are summarized in FIGS. 8A-B.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### Numbered Embodiments of the Disclosure

Other subject matter contemplated by the present disclosure is set out in the following numbered embodiments:
1. A method for processing extracellular vesicles, comprising: a) contacting a biological sample comprising a plurality of extracellular vesicles with a cargo molecule and a detergent to form a detergent-mixture solution; and b) removing the detergent from the detergent-mixture solution to obtain a plurality of cargo-loaded extracellular vesicles.
2. The method of embodiment 1, wherein the contacting the biological sample with the cargo molecule and the detergent comprises adding the cargo molecule and the detergent simultaneously or sequentially.
3. The method of embodiment 1 or 2, wherein the contacting the biological sample with the cargo molecule and the detergent comprises adding the cargo molecule prior to adding the detergent, or vice versa.
4. The method of any one of embodiments 1-3, wherein the detergent comprises a surfactant.
5. The method of embodiment 4, wherein the surfactant is a non-ionic surfactant.
6. The method of embodiment 4, wherein the surfactant comprises a hydrophilic group and a hydrophobic group.
7. The method of embodiment 6, wherein the hydrophilic group comprises a polyethylene oxide chain.
8. The method of embodiment 4, wherein the surfactant is 2-[4-(2,4,4-trimethylpentan-2-yl)phenoxy] ethanol.
9. The method of embodiment 4, wherein the surfactant is dodecyloctaglycol or octaethylene glycol monododecyl ether.
10. The method of any one of embodiments 1-9, wherein the cargo molecule comprises an active pharmaceutical ingredient (API).
11. The method of any one of embodiments 1-10, wherein the cargo molecule comprises a small molecule therapeutics.
12. The method of any one of embodiments 1-11, wherein the cargo molecule comprises a polypeptide, protein, lipid, nucleic acid, carbohydrate, lipid, metabolite, or any combinations thereof.
13. The method of embodiment 12, wherein the nucleic acid comprises DNA.
14. The method of embodiment 12, wherein the nucleic acid comprises peptide nucleic acids (PNAs).
15. The method of embodiment 12, wherein the nucleic acid comprises RNA.
16. The method of embodiment 15, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (IncRNA).
17. The method of embodiment 12, wherein the protein comprises an antibody or enzyme.
18. The method of any one of embodiments 1-17, wherein the cargo molecule comprises antisense oligonucleotide.
19. The method of any one of embodiments 1-18, wherein the cargo molecule comprises morpholino oligomer.
20. The method of any one of embodiments 1-19, wherein the cargo molecule comprises one or more components of a gene editing system.
21. The method of embodiment 20, wherein the gene editing system is selected from the group consisting of CRISPR/Cas, zinc finger nuclease, transcription, and activator-like effector nuclease (TALEN).
22. The method of any one of embodiments 1-21, wherein the plurality of extracellular vesicles comprises exosomes, microvesicles, apoptotic bodies, or any combination thereof.
23. The method of embodiment 22, wherein the plurality of extracellular vesicles comprises exosomes.
24. The method of any one of embodiments 1-23, comprising removing the detergent from the detergent-mixture solution using dialysis or ultra-centrifugation.
25. The method of any one of embodiments 1-24, wherein removing the detergent from the detergent-mixture solution comprises contacting the detergent-mixture solution with a detergent-removal agent.
26. The method of embodiment 25, wherein the detergent-removal agent comprises a nonpolar polymeric adsorbent.
27. The method of embodiment 26, wherein the nonpolar polymeric adsorbent comprises a polystyrene bead.
28. The method of embodiment 26, wherein the nonpolar polymeric adsorbent comprises a SM-2 resin.
29. The method of any one of embodiments 1-28, further comprising obtaining the plurality of extracellular vesicles from a cell culture medium.
30. The method of embodiment 29, wherein the cell culture medium is derived by growing a plurality of cultured cells.
31. The method of embodiment 30, wherein the plurality of cultured cells comprises stem cells, human embryonic kidney 293 (HEK 293) cells, HEK 293T cells, or any combinations thereof.
32. The method of embodiment 31, wherein the stem cells comprise keratinocyte stem cells.
33. The method of any one of embodiments 1-32, further comprising purifying the plurality of extracellular vesicles by centrifugation.
34. A kit for processing extracellular vesicles, comprising the detergent and detergent-removal agent of any one of embodiments 1-33.
35. The kit of embodiment 34, further comprising the cargo molecule.
36. A composition comprising a plurality of cargo-loaded extracellular vesicles generated by any of the embodiments 1-35.
37. The composition of embodiment 36, further comprising a pharmaceutically acceptable excipient.

The various embodiments described above can be combined to provide further embodiments. All of the U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified, if necessary to employ concepts of the various patents, application and publications to provide yet further embodiments.

What is claimed is:

1. A method for processing extracellular vesicles, comprising:
a) contacting a biological sample comprising a plurality of extracellular vesicles with a cargo molecule and a detergent to form a detergent-mixture solution; and
b) removing the detergent from the detergent-mixture solution to obtain a plurality of cargo-loaded extracellular vesicles.

2. The method of claim 1, wherein the contacting the biological sample with the cargo molecule and the detergent comprises adding the cargo molecule and the detergent simultaneously or sequentially.

3. The method of claim 1 or 2, wherein the contacting the biological sample with the cargo molecule and the detergent comprises adding the cargo molecule prior to adding the detergent, or vice versa.

4. The method of any one of claims 1-3, wherein the detergent comprises a surfactant.

5. The method of claim 4, wherein the surfactant is a non-ionic surfactant.

6. The method of claim 4, wherein the surfactant comprises a hydrophilic group and a hydrophobic group.

7. The method of claim 6, wherein the hydrophilic group comprises a polyethylene oxide chain.

8. The method of claim 4, wherein the surfactant is 2-[4-(2,4,4-trimethylpentan-2-yl)phenoxy] ethanol.

9. The method of claim 4, wherein the surfactant is dodecyloctaglycol or octaethylene glycol monododecyl ether.

10. The method of any one of claims 1-9, wherein the cargo molecule comprises an active pharmaceutical ingredient (API).

11. The method of any one of claims 1-10, wherein the cargo molecule comprises a small molecule therapeutics.

12. The method of any one of claims 1-11, wherein the cargo molecule comprises a polypeptide, protein, lipid, nucleic acid, carbohydrate, lipid, metabolite, or any combinations thereof.

13. The method of claim 12, wherein the nucleic acid comprises DNA.

14. The method of claim 12, wherein the nucleic acid comprises peptide nucleic acids (PNAs).

15. The method of claim 12, wherein the nucleic acid comprises RNA.

16. The method of claim 15, wherein the RNA is selected from the group consisting of mRNA, small interfering RNA (siRNA), short hairpin RNA (shRNA), piwi-interacting RNA (piRNA), small nucleolar RNAs (snoRNAs), antisense RNA, microRNA (mi-RNA), and long non-coding RNA (IncRNA).

17. The method of claim 12, wherein the protein comprises an antibody or enzyme.

18. The method of any one of claims 1-17, wherein the cargo molecule comprises antisense oligonucleotide.

19. The method of any one of claims 1-18, wherein the cargo molecule comprises morpholino oligomer.

20. The method of any one of claims 1-19, wherein the cargo molecule comprises one or more components of a gene editing system.

21. The method of claim 20, wherein the gene editing system is selected from the group consisting of CRISPR/Cas, zinc finger nuclease, transcription, and activator-like effector nuclease (TALEN).

22. The method of any one of claims 1-21, wherein the plurality of extracellular vesicles comprises exosomes, microvesicles, apoptotic bodies, or any combination thereof.

23. The method of claim 22, wherein the plurality of extracellular vesicles comprises exosomes.

24. The method of any one of claims 1-23, comprising removing the detergent from the detergent- mixture solution using dialysis or ultra-centrifugation.

25. The method of any one of claims 1-24, wherein removing the detergent from the detergent-mixture solution comprises contacting the detergent-mixture solution with a detergent-removal agent.

26. The method of claim 25, wherein the detergent-removal agent comprises a nonpolar polymeric adsorbent.

27. The method of claim 26, wherein the nonpolar polymeric adsorbent comprises a polystyrene bead.

28. The method of claim 26, wherein the nonpolar polymeric adsorbent comprises a SM-2 resin.

29. The method of any one of claims 1-28, further comprising obtaining the plurality of extracellular vesicles from a cell culture medium.

30. The method of claim 29, wherein the cell culture medium is derived by growing a plurality of cultured cells.

31. The method of claim 30, wherein the plurality of cultured cells comprises stem cells, human embryonic kidney 293 (HEK 293) cells, HEK 293T cells, or any combinations thereof.

32. The method of claim 31, wherein the stem cells comprise keratinocyte stem cells.

33. The method of any one of claims 1-32, further comprising purifying the plurality of extracellular vesicles by centrifugation.

34. A kit for processing extracellular vesicles, comprising the detergent and detergent-removal agent of any one of claims 1-33.

35. The kit of claim 34, further comprising the cargo molecule.

36. A composition comprising a plurality of cargo-loaded extracellular vesicles generated by any of the claims 1-35.

37. The composition of claim 36, further comprising a pharmaceutically acceptable excipient.
